(19) United States
(12) Patent Application Publication
(10) Pub. No.: US 2005/0244447 A1
(43) Pub. Date: Nov. 3, 2005

(54) Novel drug dosing regimen

(76) Inventor: David Duncan Heath, Upper Hutt (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/517,077

(22) PCT Filed: Jun. 5, 2003
(86) PCT No.: PCT/NZ03/00115

§ 371 Date: Jun. 1, 2005

(30) Foreign Application Priority Data
Jun. 5, 2002 (NZ) 519363

(51) Int. Cl. A61F 13/00

(52) U.S. Cl. 424/422; 424/93.1

(57) ABSTRACT

A process for administering to an animal progressively increasing doses of one or more biologically active agents which are released over a predetermined period of time from a delivery means which is administered to an animal on a single occasion.

### TECHNICAL FIELD

This invention relates to a novel drug dosing regimen. More particularly it relates to a novel immunisation/vaccination regimen and a delivery means therefore, though this should not be seen as limiting for it is anticipated such a drug dosing regimen could also be used for the delivery of biologically active agents other than vaccines, such as hormones, neutraceuticals, vitamins and trace-elements.

### BACKGROUND ART

Current immunisation/vaccination regimens usually entail an initial immunisation followed by one or more booster immunisations at defined intervals. This regimen appears to be necessary for the induction of antigen-specific memory lymphocytes, particularly memory B lymphocytes.

Typical regimens for both humans and animals are, for example, immunisation at 0, 4 and 26 or 52 weeks, although immune responses vary significantly with immunisation regimens and current regimens have been designed to optimise antibody production.

In the immunisation of farm animals, a vaccination regimen of 0 and 4 weeks, for example, would involve the mustering and re-mustering of animals which is very time consuming, expensive and difficult to ensure that every animal is immunised at the right time.

Accordingly it would be desirable to have an alternative immunisation/vaccination regimen which would provide effective immunisation without the need for repeated booster immunisations. Such a drug dosing regime would also be useful for the delivery of biologically active agents other than vaccines, such as hormones, neutraceuticals, vitamins and trace-elements.

A number of techniques have previously been published which describe the delivery of a single dose of antigen sufficient to generate primary immunisation, through the controlled release of antigen over an extended period.

For example, Gupta et al. (Developments in Biological Standardization. 1998, vol. 92, pp 63-78) describes the use of biodegradable polymer microspheres as vaccine adjuvants and delivery systems. In this article, Gupta describes how such microspheres form a depot at the injection site, allowing the slow release of antigen over an extended period and thus maintaining high levels of antibodies during this time.

Whilst useful for generating a primary immune response, such controlled release of antigen as described by Gupta does not detail increasing doses of drugs of the mimicking of a natural episode of infection.

U.S. Pat. No. 6,074,673 also describes an implantable slow-release device for use in allergy desensitisation systems. This document merely details the controlled release of antigen over a period of time, rather than mimicking a natural episode of infection. Instead, this document is directed to the use of increasing drug dosages or the desensitisation of a patient to a given antigen.

WO 01/07079 describes a device which provides an initial release of antigenic material, followed by a steady, slower release over a prolonged period of time. A secondary “boosted” release, above that of the base level, of antigenic material can occur some time after the initial dose, “typically taking the place of the booster shot in conventional vaccination techniques”.

WO 01/07079 further describes how “a higher release rate may be released to act as a booster”, though it is not defined whether this ‘booster’ is intended to exceed the rate of initial release. In fact, it is stated that care should be taken that the release amount does not result in desensitising the subject to released antigenic material. As such, this document teaches away from increased rates of release of drugs, or the mimicking of a natural episode of infection.

U.S. Pat. No. 6,010,492 describe devices capable of subsequent doses of a given drug, to boost the falling levels of the drug in a subject in order to maintain efficacious levels. However, once again this document does not describe the delivery of increasing doses of a drug, but rather the delivery of the same set dose before the circulating drug level falls to zero, thus increasing the total level of the drug in a subject. There is no mention in this document of increasing doses, or the mimicking of a natural episode of infection.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a process for administering to an animal at least three progressively increasing doses of one or more biologically active agents which are released over a predetermined period of time from a delivery means which is administered to an animal on a single occasion.

Preferably said one or more biologically active agents are selected from the list of antibiotics, anthelmintics, peptides, proteins, carbohydrates, DNA, RNA, hormones, neutraceuticals, vitamins, trace-elements, immunising agents or any combination thereof.

The biologically active agents may optionally comprise an adjuvant and/or a pharmaceutically acceptable carrier.

Preferably the progressively increasing doses comprise sequentially doubling doses of biologically active agent or sequentially increasing doses such as 25, 50, 75, 100 or 4, 8, 32, 150 active units. The doses are chosen so as to elicit a desired response and are administered over a predetermined period of tie from hours, days, weeks, and months or any combination thereof.

Preferably said one or more biologically active agents is an immunising agent and wherein said process comprises the administration to said animal of progressively increasing doses of one or more immunising agents which are released over a predetermined period of time from a delivery means which is administered to an, animal on a single occasion.

Preferably said one or more biologically active agents are selected from the list of antibiotics, anthelmintics, peptides, proteins, carbohydrates, DNA, RNA, hormones, neutraceuticals, vitamins, trace-elements, immunising agents or any combination thereof.

Preferably said one or more biologically active agents comprise an adjuvant and/or a pharmaceutically acceptable carrier.

Preferably the biologically active agent delivery composition comprises means to enable the delivery of the progressively increasing doses comprise sequentially doubling doses of biologically active agents, or sequentially increasing doses such as 25, 50, 75, 100 or 4, 8, 32, 150 active units.

Preferably said one or more biologically active agents is an immunising agent and wherein said delivery composition comprises means to enable progressively increasing doses of one or more immunising agents to be released over a predetermined period of time from a delivery means which is administered to an animal on a single occasion.

Preferably said one or more immunising agents comprise an antigen/vaccine or combination of antigens/vaccines.

Preferably the antigen/vaccine is selected from molecules that will induce protective immunity against a disease causing organism, or functional immunity or any combination thereof.

Preferably, the progressively increasing doses comprise sequentially doubling doses of antigen/vaccine, or sequentially-increasing doses such as 25, 50, 75, 100 μg.

This novel immunisation/vaccination regimen mimics the growth of bacteria during an episode of infection and the natural antibody response thereto.

The doses of the progressively increasing doses of antigen are chosen so as to elicit a favourable antibody response which includes the production of both high-affinity antibodies and antigen-specific memory lymphocytes.

Preferably, the progressively increasing doses of antigen are in the range of from 0.1 μg to 1000 μg.

Preferably, the predetermined period of time at which the one or more drugs are administered is selected from hours, days, weeks or months or any combination thereof.

The delivery means preferably comprises an immunising agent delivery composition which comprises means to enable progressively increasing doses of one or more immunising agents to be released over a predetermined period of time therefrom when said delivery means is administered to an animal on a single occasion.

The present invention further provides an unloaded delivery means comprising an unloaded immunising agent delivery composition which comprises means to enable at least three progressively increasing doses of one or more immunising agents to be released over a predetermined period of time once said immunising agent is loaded into said immunising agent delivery composition and when said delivery means is administered to an animal on a single occasion.

Preferably, the delivery composition comprises means to enable the delivery of one dose of said one or more antigens within hours/days/weeks/months of its administration and means to enable the delivery of further progressively increasing doses of the same or different antigens hours/days/weeks or months later.

The progressively increasing doses of said one or more immunising agents are preferably in the range of from 0.1 μg to 1000 μg.

Preferably the delivery composition comprises said one or more immunising agents that are selected from molecules that will induce protective immunity against a disease causing organism, or functional immunity or any combination thereof.

In one alternative, the delivery composition comprises two or more types of microspheres or microparticles, each type of microsphere or microparticle containing a different dose of one or more antigens and comprising biodegradable material which will degrade over a known time period so that the lowest dose of antigen is released from a first type of microsphere or microparticle at a set time after administration, followed by the next highest dose of antigen at the next predetermined time etc.

In another alternative, the composition may be located within known or novel drug delivery devices, for example, the drug delivery device may be of a multi-compartmental capsule type containing progressively increasing doses of one or more immunising agents within the compartments, said device comprising an outer wall made of a biodegradable substance which degrades over a pre-set period of time to release the smallest dose of drug(s), and one or more inner compartmental walls made of the same or different material that degrade over a longer period of time to release progressively increasing pulses of the bioactive ingredient.

The biodegradable material of the outer wall may be selected from the group comprising cholesterol/lecithin, polylactide and/or polyglycolide copolymers, one or more of a number of cellulose polymers, polyacrylic acid, polymethylmethacrylate, cross-linked polyacrylic acid, polycaprolactone, polyvinylpyrrolidine, polyvinylalcohol, polyethylene glycol, agarose, DEAE dextran microspheres, starch microspheres and/or albumin microspheres, gelatine microspheres or any other compound or combination thereof.

The biodegradable material of the inner compartmental walls may be selected from the above named compounds.

The pre-set period of time within which the outer wall and inner compartmental walls degrade may be selected from hours, days, weeks or months.

Alternatively, the drug delivery device may comprise a capsule having an osmotic pump therein as is known from ALZET Technical Information Services, ALZA Corporation, 950 Page Mill Road P.O. Box 10950, Palo Alto, Calif. 94303-0802, USA.

Other known drug delivery devices may also be used as vehicles to deliver the novel drug dosing regimen of the present invention.

Alternatively, the drug delivery device may comprise a bioerodable device that releases progressively increasing amounts of antigens as it erodes.

The progressively increasing doses of the one or more immunising agents may be administered to an animal by way of injection, ingestion or implantation.

The administration, i.e. injection, ingestion or implantation, of the immunising agents according to the novel dose regimen of the present invention preferably takes place shortly after birth of an animal, or when maternally-derived antibody has decreased sufficiently for the young animal to be able to respond to the vaccination, and provides immunity without the need for further booster administration.

The present invention further provides a drug delivery device comprising a biologically active agent delivery composition according to the invention.

The drug delivery device is selected from the group comprising, multi-compartmental capsules and capsules having as osmotic pump therein or any other known drug delivery device to which the novel drug delivery regimen is incorporated.

The delivery means comprises a biologically active agent delivery composition which comprises means to enable progressively increasing doses of one or more biologically active agents to be released over a predetermined period of time therefrom, when said delivery means is administered to an animal on a single occasion.

A delivery composition as is described above for the biologically active agent delivery composition except that the composition is either loaded or unloaded with one or more biologically active agent other than an immunising agent.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### Definitions

Eg95; A vaccine molecule which protects sheep against acquisition of the cysts of Echzinococcus granulosus (hydatid Disease).

GST Eg95; The fusion partner of the recombinant fusion protein is glutathione-S-transferase (GST) from Schistosoma japonicum.

PIRS; Progressively Increasing Release System

Quil A; A purified extract from the bark of a South American tree (Quillaia saponaria) which is used as an adjuvant for the vaccine Eg95.

### BRIEF DESCRIPTION OF DRAWINGS

The present invention will become apparent from the following description which is given by way of example only and with reference to the figures of the accompanying drawings in which:

FIG. 1 shows the antibody response to conventional immunisation regimen in sheep having 50 μg of Eg95 vaccine administered at 2 months and 3 months of age and a booster at 8 months of age, compared to control sheep who received no vaccine.

FIG. 2 shows the antibody response over time to a single injection of 50 or 100 μg of Eg95 vaccine in control sheep compared to an injection of an osmotic pump drug delivery device containing a single dose of 50 or 100 μg of Eg95 vaccine.

FIG. 3 shows individual data for four sheep injected daily with progressively increasing doses of Eg95 recombinant antigen and Quil A adjuvant (PIRS) in comparison to four sheep receiving two injections one month apart of 50 μg Eg95 and Quil A (control).

FIG. 4 shows the combined data of the sheep of FIG. 3 with nineteen antibody measurements taken over 18 months.

FIG. 5 shows the antibody response of sheep to progressively increasing doses of GST Eg95 vaccine, 25 μg, 50 μg, 75 μg and 100 μg administered at weekly intervals (PIRS) compared to two control groups, “control +” which were given two doses of 125 μg GST Eg95 four weeks apart and “control −” which were given Quil A adjuvant only in two doses at four weeks apart.

FIG. 6 shows the antibody response of sheep to progressively increasing doses of GST Eg95 vaccine, 4 μg, 8 μg, 32 μg and 150 μg administered at weekly intervals with a standard amount of Quil A (Group 1) or with increasing amounts of Quil A (Group 2) compared to two control groups given two doses of 50 μg GST Eg95 being either urea depleted (Group 3) or non-urea depleted (Group 5) and standard amounts of Quil A four weeks apart and a negative control (Group 4) which had no injections.

FIG. 7 shows the antibody response of cattle to progressively increasing doses of Clostridium novyi administered at weekly intervals with a standard amount of Quil A (Group 1) or with increasing amounts of Quil A (Group 2), a normal vaccination procedure of 2 injections given 28 days apart with standard levels of Quil A each time (Group 3) and progressively increasing doses administered daily for 21 days (Group 4).

FIG. 8 shows the antibody response of cattle to progressively increasing doses of Clostridium tetani administered at weekly intervals with a standard amount of Quil A (Group 1) or with increasing amounts of Quil A (Group 2), a normal vaccination procedure of 2 injections given 28 days apart with standard levels of Quil A each time (Group 3) and progressively increasing doses administered daily for 21 days.

### EXAMPLE ONE

### Experimental Details

1. Conventional Vaccination Regimen

Two injections were given, one month apart, subcutaneously in the neck region. The vaccine consisted of 50 μg of Eg95/GST fusion protein active ingredient and 1 mg of Quil A (Superfos Biosector, Denmark), which was reconstituted from lyophilised batches of 50 doses. A new batch was reconstituted each time. Vaccination was repeated 6 months after the second vaccination, to promote an anamnestic response. The vaccine (China 5000) was formulated on Dec. 11, 1996 and made from inclusion bodies grown on Jan. 9, 1996. The formulation for the China 5000 Vaccine used in the trials is as follows:
Total Protein=5.4 mg/mL
% Eg95 protein=22.3%
Total Eg95 protein=5.4×0.223=1.2 mg/mL

50 doses were made up in 10 mL at 50 μg /mL=2500 μg Eg95=2.08 mL of solubilised Inclusion bodies+7.92 mL formulation buffer+50 mg of Quil A.

| Formulation Buffer | PBS pH 6.8 |
|---|---|
| Na₂HPO₄.12H₂O | 4.656 g/L |
| NaH₂PO₄.2H₂O | 1.092 g/L |
| NaCl | 8.5 g/L |

Phenonip (NIPA Laboratories Ltd) 2.5 mL/L
Water for Manufacturing to 1 litre.

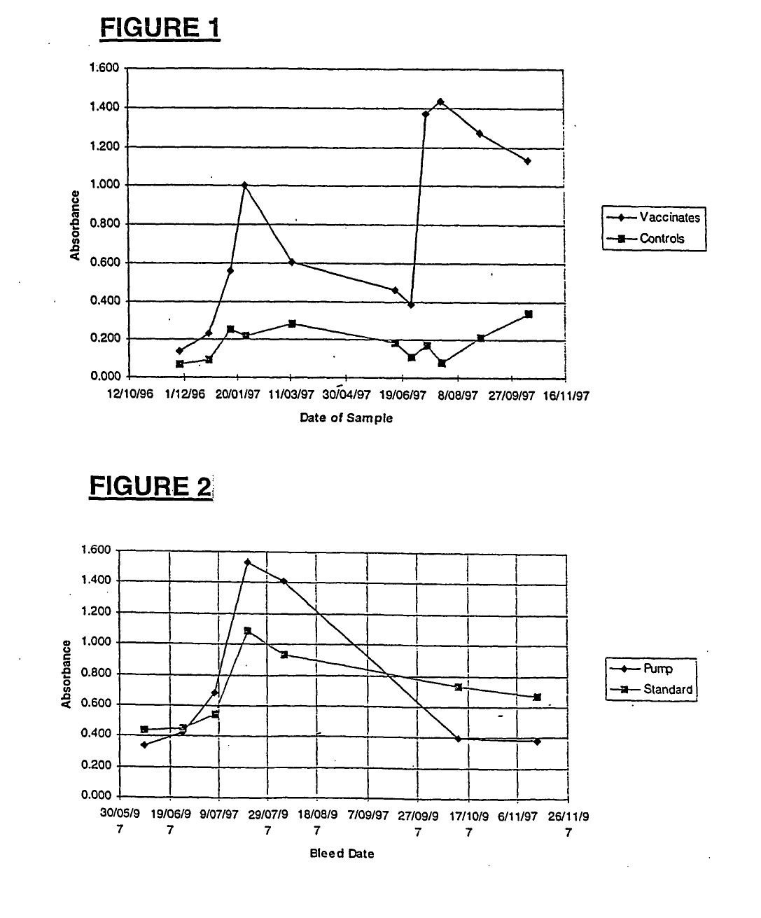

Sheep were bled regularly from the jugular vein, using “vacutainer” equipment, and sera was stored at −20° C. until required for testing. Absorbances of the ELISA test are shown in FIG. 1. The antigen used in the ELISA was Eg95/HIS. The polyhistidine fusion protein (Quiagen) did not react with antibody generated against the GST portion of the vaccinating molecule.

### Results

The conventional protocol for delivering vaccine is two injections, given approximately one month apart. Ideally, the antibody response to the first injection has begun to wane before the second injection—the second injection is then more successful in stimulating clonal expansion of antibody-forming cells. A third injection given 3-12 months later causes the so-called anamnestic response, where increased clonal expansion occurs and long-lived antibody-forming cells are stimulated. FIG. 1 demonstrates this principle.

2. Osmotic Pump Trial

The aim of this trial was to determine what level of continuous release of antigen and adjuvant was necessary to maintain a level of antibody in serum that was equivalent to that stimulated by conventional vaccination technology. Based on this information, a slow-release delivery device would be devised to perform the same function as the osmotic pumps.

ALZET osmotic pumps were chosen that delivered 2.5 μl/hour for 28 days, and after filling with a suitable dosage of antigen, they were surgically-implanted subcutaneously in the shoulder region. After 28 days, a new pump was inserted into an area close to the original pump, and the original was withdrawn.

Four 6-month-old lambs were vaccinated conventionally with either 50 or 100 μg of Eg95/GST with Quil A as shown in Table 1, and another 4 were implanted with pumps that delivered the same amount of antigen as above, but continuously released over 28 days. At the end of the 28 days, the conventional vaccination was repeated, and the pumps were replaced. After a further 28 days, the second series of pumps were withdrawn. Blood was collected and serum stored for ELISA analysis, at fortnightly intervals, and at the 2 and 3 months after the end of the vaccination period.

TABLE 1

| Group | Sheep | Vacc. | Route | Quil A | Eg95 | PBS | Volume |
|---|---|---|---|---|---|---|---|
| 1 | 4966 | inject | s/c | 1000 μg | 50 μg | — | 0.2 ml |
| 1 | 4970 | inject | s/c | 1000 μg | 50 μg | — | 0.2 ml |
| 2 | 4965 | inject | s/c | 2000 μg | 100 μg | — | 0.4 ml |
| 2 | 4985 | inject | s/c | 2000 μg | 100 μg | — | 0.4 ml |
| 3 | 4980 | pump | s/c | 1000 μg | 50 μg | 2.1 ml | 2.3 ml |
| 3 | 4971 | pump | s/c | 1000 μg | 50 μg | 2.1 ml | 2.3 ml |
| 4 | 4968 | pump | s/c | 2000 μg | 100 μg | 1.9 ml | 2.3 ml |
| 4 | 4986 | pump | s/c | 2000 μg | 100 μg | 1.9 ml | 2.3 ml |

### Results

FIG. 2 demonstrated that continuous release of antigen and adjuvant from an osmotic pump can cause antibodies to rise to levels above that achieved with conventional vaccination. However, the cessation of antigen release resulted in a return to zero levels of antibody production. The animals receiving a continuous priming dose of antigen are fully-primed for a secondary response (data not shown). However, the aim of these experiments was to devise single-shot antigen presentation.

3. Progressively Increasing Release System for Vaccines (PIRS I and PIRS II)

There is no record of research that attempts to duplicate a proliferative disease, which tends to provide antigen exponentially to the host until after the host has fully-responded and destroyed the pathogen. The aim of a regimen of progressively increasing release of antigen was investigated as a means of achieving the high titres normally resulting from the conventional 2 injections at, for example, 0 and 1 month of vaccination. A successful demonstration using carefully-graded daily injections will allow for the duplication of this style of antigen release in a biodegradable pellet of novel constructions.

Protocol:

Arithmetically Increasing Vaccination.

Preparation of the vaccine: (SC1300, His antigen solubilised in sarkosyl)

The antigen for the vaccine was obtained from fermentation batch, Nov. 14, 1996. Inclusion bodies isolated from the culture were solubilised in 1.5% sarkosyl. The original concentration of the sarkosyl preparation had been approximately determined from page gel analysis to be 7.9 mg/ml. To obtain a 50 μg/ml vaccine the sarkosyl preparation needed to be diluted by a factor of 158. It was decided that a 0.5 ml dose would be given and therefore the vaccine concentration was made up to 100 μg/ml in formulation buffer. PBS, pH6.8: Na₂BPO₄.12H₂O (4.656 g/L); Na₂H₂PO₄.2H₂O (1.092 g/L); NaCl (8.5 g/L); phenonip (2.5 mL/L); in d.H₂O.

Dilution of Sarkosyl Preparation:

254 μl of the sarkosyl solubilised inclusion bodies, was added to 20 mls of formulation buffer.

Quil A was also added to the vaccine at a concentration of 2 mg/ml.

From the 100 μg/ml vaccine 2.5 mls was removed and added to another 2.5 mls of formulation buffer. This was to prepare a vaccine with antigen concentration of 50 μg/ml. This double dilution procedure was repeated until a vaccine was prepared with antigen concentration of 0.000019 μg/ml.

Vaccination Procedure:

4 sheep were injected daily with arithmetically-increasing (double dilution) doses of Eg95 recombinant antigen and Quil A as adjuvant (PIRS) for 20 days as follows:

On Day 1 0.000095 μg of antigen, finishing on Day 20 with 50 μg of antigen and 1 mg Quil A (see Table 2 below).

(The Quil A was doubly diluted also, by making up 200 μg of antigen and 4 mg of Quil A, and doubly diluting this mix 19 times). Four control sheep received 50 μg Eg95 and 1 ml Quil A on Days 1 and 30. Serum was taken from all sheep weekly to monitor antibody responses. Responses were tested against Eg95 using the ELISA test. Monthly serum samples were taken and monitored for the following 18 months.

TABLE 2

| Day No. | Dose (μg) |
|---|---|
| 1 | 0.000095 |
| 2 | 0.00019 |
| 3 | 0.00038 |
| 4 | 0.00076 |
| 5 | 0.0015 |
| 6 | 0.003 |
| 7 | 0.006 |
| 8 | 0.012 |
| 9 | 0.024 |
| 10 | 0.048 |
| 11 | 0.096 |
| 12 | 0.192 |
| 13 | 0.38 |
| 14 | 0.78 |
| 15 | 1.56 |
| 16 | 3.12 |
| 17 | 6.24 |
| 18 | 12.5 |
| 19 | 25.0 |
| 20 | 50 |

### Results

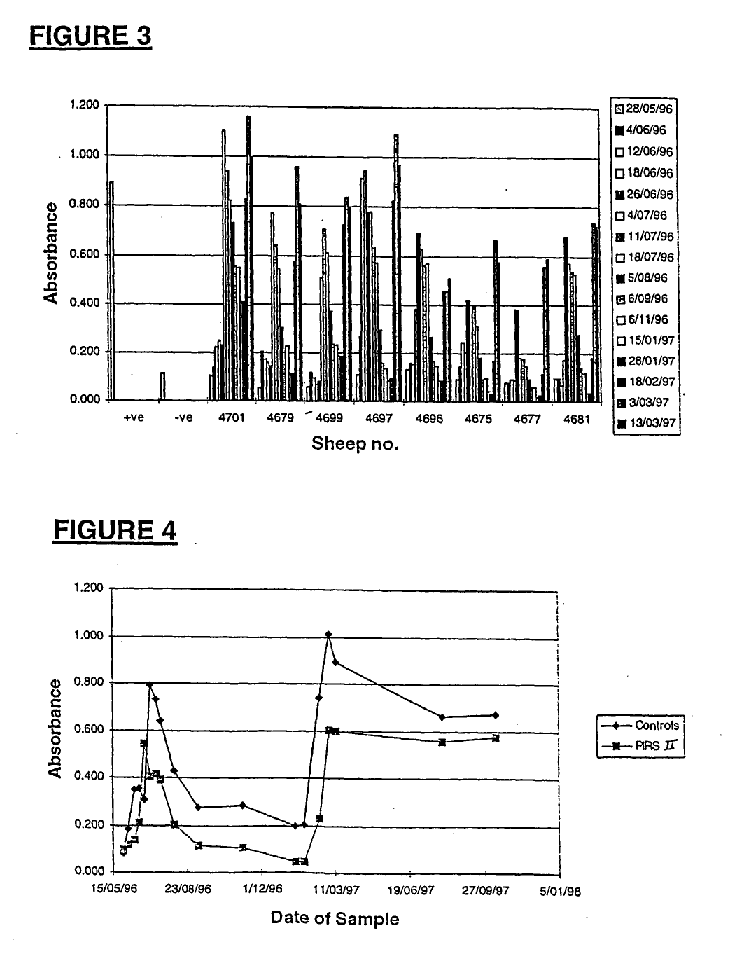

FIGS. 3 and 4 compare the results of conventional vaccination with a progressively increasing daily exposure of antigen to sheep. Sheep receiving conventional vaccination were numbered 4701, 4679, 4699 and 4697. Sheep receiving the simulation of progressively-increasing release of antigen (PIRS I and II) were numbered 4696, 4675, 4677, 4681. ELISA results showed antibody titres had dropped in the PIRS vaccinated sheep.

It was decided to boost all the sheep, both controls and PIRS sheep, with a single vaccination of 50 μg Eg95 and 1000 μg Quil A.

The vaccine used was formulated on the Oct. 11, 1995 as part of a batch of vaccine. The striking similarity of the antibody responses between the two groups indicates that PIRS is likely to be as effective as conventional vaccination in stimulating a prolonged response and an anamnestic response. Possibly one more injection should have been added to the PIRS to raise the antibody levels to those of the conventional vaccination, especially as following a booster injection 6 months later, it appears that the height of the initial antibody absorbance may have a significant influence on the height of any subsequent anamnestic response.

4. Progressively Increasing Release System for Vaccines (PIRS III)

This trial was carried out to simulate the effect of pulse releases of increasing doses of the vaccine GST Eg95 on the antibodies titre of sheep.

The vaccine used for the trial was formulated on the Dec. 11, 1996, and was made from the fermentation batch grown on the Sep. 17, 1996. The vaccine was formulated as described above.

Details of all vaccines and new formulations are described in the “Echinococcus granulosus” Vaccine Production Books available at AgResearch, Wallaceville upon request.

Protocol:

The trial was designed as follows:

Group 1 (5 sheep) received 25 μg, 50 μg, 75 μg and 100 μg of GST Eg95 at weekly intervals (PIRS).

Group 2 (5 sheep) received 2 doses of 125 μg GST Eg95 four weeks apart (control +).

Group 3 (5 sheep) received 2 doses of 1 mg Quil A, four weeks apart (control −).

### Results

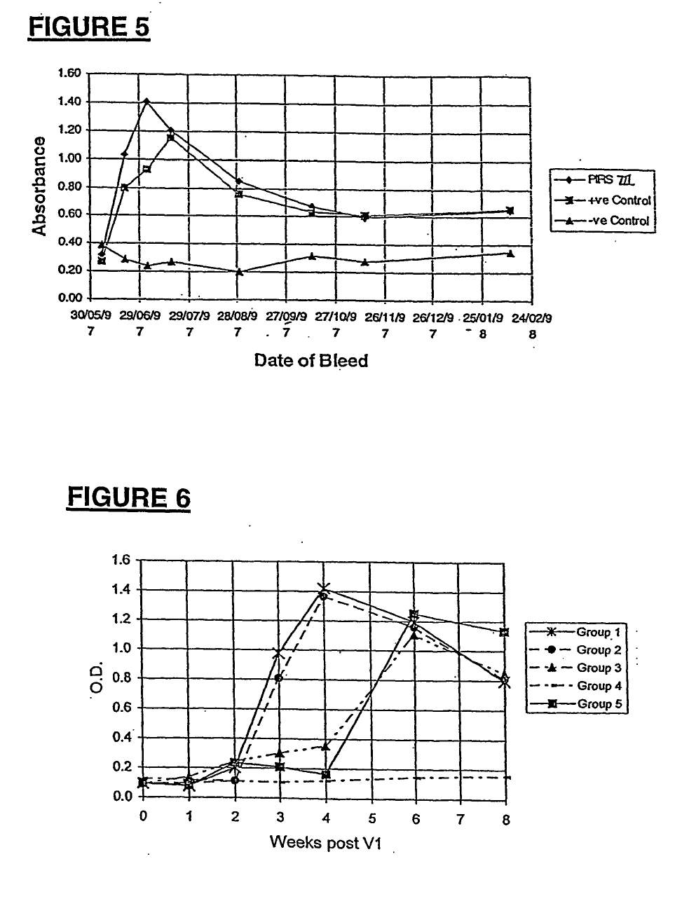

PIRS III was designed to lend itself to effective manufacture. As mentioned above, the progressively-increasing doses were given on 4 occasions, each one week apart. FIG. 5 shows that the PIRS III was clearly more effective in stimulating an early antibody response compared to conventional vaccination, and that the eventual outcome of both vaccination regimes were similar. There is clearly an advantage for disease control if an early and rapid response to the vaccine can be stimulated.

5. Progressively Increasing Release System for Vaccines (PIRS IV)

The trial was carried out to establish whether a conventional protocol of two injections could be improved by a progressively increasing release system (PIRS) protocol of four injections.

The vaccine (GST Eg95) used for Groups 1-3 of the trial was Antigen #HYD/024 which comprised:
Total protein=4.1 mg/mL
Eg95 protein=25% of total protein by densitometry=1 mg/mL

Using the stirred cell and a PM 10 membrane the antigen was concentrated and rediluted 4× using glycine buffer to remove the urea.

1 mL of HYD/024 contains 1 mg Eg95∴5 mL of HYD/024 contains 5 mg Eg95=100 doses at 50 μg/dose. The antigen was aliquoted in 5 mL amounts and freeze-dried without Quil A. This urea-depleted antigen was used for Groups 1, 2 and 3.

The vaccines were made up into 15 ml aliquots and frozen, except for Day zero.

The vaccine for Group 5 was made from HYD/024 starting material but was non-urea depleted.

Protocol:

The trial was designed as follows:

Group 1 (10 sheep) received 4 μg, 8 μg, 32 μg and 150 μg of urea depleted GST Eg95 and 0.5 mg of Quil A at weekly intervals.

Group 2 (10 sheep) received 4 μg, 8 μg, 32 μg and 150 μg of urea depleted GST Eg9S and 0.04 mg, 0.08 mg, 0.32 mg and 1.50 mg Quil A at weekly intervals.

Group 3 (10 sheep) received 2 doses of 50 μg urea depleted GST Eg95 and 1 mg Quil A four weeks apart.

Group 4 (10 sheep) received no injections (negative control).

Group 5 (10 sheep) received 2 doses of 50 μg non-urea depleted GST Eg95 and 1 mg Quil A four weeks apart.

All sheep were artificially challenged with live E. granulosus eggs, three months after the second vaccination of Groups 3 and 5, and the subsequent necropsy examined to compare the levels of protection. The level of immunity was also determined from the antibody levels which were measured from blood samples taken from all groups from Day 0 and weeks 1, 2, 3, 4, 6 and 8.

### Results

FIG. 6 shows that the PIRS IV (Groups 1 and 2) was clearly more effective in stimulating an early antibody response at four weeks compared to conventional vaccination (Groups 3 and 5) where the maximum response was not observed until six weeks. Further, the trial shows that the eventual outcome of both vaccination regimes was similar. Results of the necropsy examination will be available to check the level of immunity provided by the vaccination regimens at the cellular level.

### Conclusion

The three PIRS systems (I and II; III and IV) look to be effective in stimulating an effective primary/secondary antibody response which is equal to the widely-separated primary and secondary responses of conventional vaccination.

The daily doubling of antigen exposure system lends itself to stimulation using a progressively-increasingly bioerodable matrix. The four progressively-increasing releases of antigen lend themselves to an erodable matrix with layers of antigen/adjuvant incorporated.

### EXAMPLE TWO

### Demonstration of PIRS in Cattle Using a 5 in 1 Clostridial Vaccine

Introduction:

It is desirable to prevent the pathology of disease by a single injection of antigen, but standard vaccination procedures require two or more-often 3 injections of antigen at intervals at least one month apart. However, it has been found that a single exposure to a proliferating infection with microorganisms can lead to prolonged immunity to reinfection.

Previous experiments with sheep, using the Eg95 recombinant hydatid vaccine and the adjuvant Quil A as described above, have shown that presenting antigen to the host in a similar manner to when the host is exposed to proliferating microorganisms, stimulates a degree of immunity similar to that stimulated by proliferating microorganisms. This progressively-increasing exposure to antigen also stimulates similar immunity to that induced by two antigenic exposures given 28 days apart.

Repeated daily or weekly injections are impractical. However, the daily doubling of antigen exposure by injection is designed to simulate the erosion of a progressively-increasingly bioerodable matrix. The four injections of progressively-increasing amounts of vaccine are designed to simulate an erodable matrix with layers of antigen incorporated therein.

### Materials and Methods

Variation between animals in immunological response have shown that 6/group is the minimum number for statistically valid comparisons to be drawn between groups.

Twenty four weaned cattle were purchased and randomly divided into 4 groups of 6. Some cattle were Charolais, and the rest were Aberdeen Angus.

Cattle were purchased on the guarantee that they had not been vaccinated against Clostridial diseases.

Cattle received subcutaneous injections of different concentrations of tetanus toxoid antigen plus Quil A adjuvant in the neck region in 2 ml/injections using aseptic procedures and a 20 g needle. Group 4 received vaccinations daily for 21 days of doubly increasing concentrations of antigen (see table below).

To achieve this, a stock of formulated vaccine containing 200 μg antigen+2 mg Quil A was made. A doubling dilution of this stock vaccine was made for Day 20 through to Day 0.

Cattle were bled from the tail vein using 10 ml vacutainers and 19 g needle.

The vaccine was one that is registered in New Zealand for normal use, so that the animals can be sold into the food chain {Ultravac 5 in 1 Vaccine (CSL) containing Clostridium peifringens type D, Cl. tetani, Cl. novyi type B. Cl. septicum [as ultrafiltered toxoids], Cl. chauvoei (as formol culture)}. This vaccine is sold in an aluminium hydroxide adjuvant. Quil A adjuvant was added in order to facilitate future freeze-drying of antigen and adjuvant which is important for making single-shot implanted delivery systems. The antibody responses of the cattle were determined using ELISA and pure Cl. tetani or pure Cl. novyi antigen.

Allocation to Groups

| | | | | | | | |
|---|---|---|---|---|---|---|---|
| Group 1 | Black | 301 | 302 | 303 | 304 | 305 | 306 |
| Group 2 | White | 101 | 102 | 103 | 104 | 105 | 106 |
| Group 3 | Orange | 501 | 502 | 503 | 504 | 505 | 506 |
| Group 4 | Yellow | 31 | 32 | 33 | 34 | 35 | 36 |

Description of Group Treatments, as shown in Table 1

Group 1: A standard dose of Quil A with each of 4 progressively-increasing injections of antigen given at weekly intervals

Group 2: Progressively increasing amounts of Quil A with each of 4 progressively-increasing injections of antigen given at weekly intervals.

Group 3: Normal vaccination procedure—2 injections given 28 days apart with 1 mg Quil A each time.

Group 4: Progressively increasing daily injections given for 21 days

TABLE 1
VACCINATION/BLEEDING REGIMEN

| | | | | | Tetanus | | |
|---|---|---|---|---|---|---|---|
| | | | Vacci- | | toxoid | | |
| Group | Date | Day | nation | Bleed | antigen (μg) | Quil A | |
| 1 | 11/11/02 | 0 | V1 | ✓ | 6 | 0.5 | mg |
| | 18/11/02 | 7 | V2 | ✓ | 12 | 0.5 | mg |
| | 25/11/02 | 14 | V3 | ✓ | 32 | 0.5 | mg |
| R | 2/12/02 | 21 | V4 | ✓ | 150 | 0.5 | mg |
| 2 | 11/11/02 | 0 | V1 | ✓ | 6 | 0.06 | mg |
| | 18/11/02 | 7 | V2 | ✓ | 12 | 0.12 | mg |
| | 25/11/02 | 14 | V3 | ✓ | 32 | 0.32 | mg |
| | 2/12/02 | 21 | V4 | ✓ | 150 | 1.5 | mg |
| 3 | 11/11/02 | 0 | V1 | ✓ | 100 | 1.0 | mg |
| | | 7 | | ✓ | | | |
| | | 14 | | ✓ | | | |
| | 9/12/02 | 28 | V2 | ✓ | 100 | 1.0 | mg |
| 4 | 11/11/02 | 0 | V1 | ✓ | 0.00019 | 0.0019 | μg |
| | 12/11/02 | 1 | V2 | | 0.00038 | 0.0038 | μg |
| | 13/11/02 | 2 | V3 | | 0.00076 | 0.0076 | μg |
| | 14/11/02 | 3 | V4 | | 0.00153 | 0.0153 | μg |
| | 15/11/02 | 4 | V5 | | 0.0031 | 0.031 | μg |
| | 16/11/02 | 5 | V6 | | 0.0061 | 0.061 | μg |
| | 17/11/02 | 6 | V7 | | 0.0122 | 0.122 | μg |
| | 8/11/02 | 7 | V8 | ✓ | 0.0244 | 0.244 | μg |
| | 19/11/02 | 8 | V9 | | 0.0488 | 0.488 | μg |
| | 20/11/02 | 9 | V10 | | 0.098 | 098 | μg |
| | 21/11/02 | 10 | V11 | | 0.195 | 1.95 | μg |
| | 22/11/02 | 11 | V12 | | 0.39 | 3.9 | μg |
| | 23/11/02 | 12 | V13 | | 0.78 | 7.8 | μg |
| | 24/11/02 | 13 | V14 | | 1.563 | 15.63 | μg |
| | 25/11/02 | 14 | V15 | ✓ | 3.125 | 31.25 | μg |
| | 26/11/02 | 15 | V16 | | 6.25 | 62.5 | μg |
| | 27/11/02 | 16 | V17 | | 12.5 | 125 | μg |
| | 28/11/02 | 17 | V18 | | 25 | 250 | μg |
| | 29/11/02 | 18 | V19 | | 50 | 500 | μg |
| | 30/11/02 | 19 | V20 | | 100 | 1000 | μg |
| | 1/12/02 | 20 | V21 | | 200 | 2000 | μg |
| | 2/12/02 | 21 | | ✓ | | | |
| | 9/12/02 | 28 | | ✓ | | | |

All cattle were bled on days 35, 42, 56 and 84

Possible Vaccine Reactions

A slight rise in temperature immediately following vaccinations containing 500 μg or more of Quil A, returning to normal within 1 to 2 days. A small injection site reaction returning to normal within 2 to 4 weeks. Both of these reactions are normal following most vaccinations.

### Results

### Vaccines Lesions in Cattle 1 Week After the Day 28 Injection

### All Lumps Had Disappeared by Day 56

| | | Height of Lesion(cm) | Area of lesion(cm) |
|---|---|---|---|
| Group 1 Black | 301 | Nil | Nil |
| | 302 | 0.5 | 1 × 1 |
| | 303 | Nil | Nil |
| | 304 | 0.5 | 3 × 3 |
| | 305 | Nil | Nil |
| | 306 | Nil | Nil |
| Group 2 White | 101 | 1.0 | 4 × 2 |
| | 102 | 0.5 | 4 × 4 |
| | 103 | Nil | Nil |
| | 104 | Nil | Nil |
| | 105 | Nil | Nil |
| | 106 | 1.0 | 2 × 4 |
| Group 3 | 501 | 0.5 | 2 × 6 |
| Orange | 502 | 1.0 | 6 × 2 |
| | 503 | Nil | Nil |
| | 504 | 0.5 | 2 × 2 |
| | 505 | Nil | Nil |
| | 506 | 1.0 | 2 × 2 |
| Group 4 | 31 | 3.0 | 4 × 6 |
| Yellow | 32 | 4.0 | 4 × 20 |
| | 33 | 2.7, 1.0 | 4 × 4, 2 × 1 |
| | 34 | 2.0, 1.0, 1.0 | 5 × 5, 3 × 3, 5 × 2 |
| | 35 | 0.5, 2.0 | 2 × 1, 5 × 4 |
| | 36 | 1.0, 0.5 | 6 × 6, 2 × 2 |

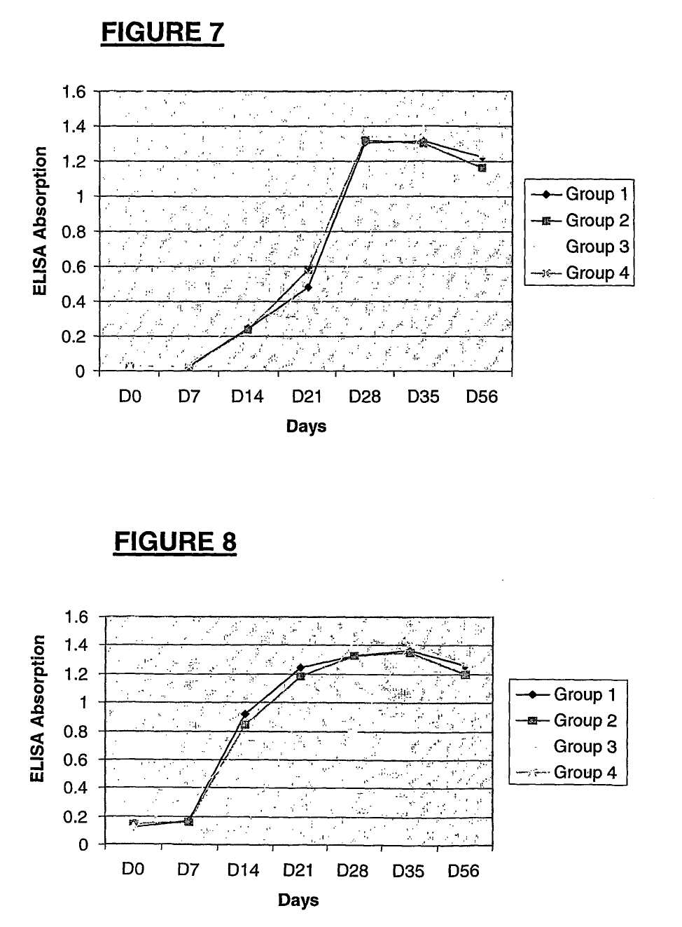

### Antibody Responses of Cattle to Progressively Increasing Doses of Clostridium novyi as Shown in FIG. 7.

| | Group 1 | Group 2 | Group 3 | Group 4 |
|---|---|---|---|---|
| D0 | 0.0235 | 0.024333 | 0.025167 | 0.026 |
| D7 | 0.0255 | 0.022333 | 0.041167 | 0.027333 |
| D14 | 0.243833 | 0.2395 | 0.342333 | 0.029 |
| D21 | 0.483333 | 0.582167 | 0.296833 | 0.5655 |
| D28 | 1.303833 | 1.317167 | 0.738667 | 1.383 |
| D35 | 1.3145 | 1.302167 | 1.385667 | 1.36 |
| D56 | 1.226833 | 1.160667 | 1.245333 | 1.239167 |

### Antibody Responses of Cattle to Progressively Increasing Doses of Clostridium tenani as Shown in FIG. 8.

| | Group 1 | Group 2 | Group 3 | Group 4 |
|---|---|---|---|---|
| D0 | 0.120333 | 0.1475 | 0.125333 | 0.107333 |
| D7 | 0.16775 | 0.160917 | 0.296667 | 0.101167 |
| D14 | 0.915583 | 0.844667 | 0.730917 | 0.108417 |
| D21 | 1.242417 | 1.181417 | 0.701667 | 0.924375 |
| D28 | 1.32325 | 1.329583 | 0.80275 | 1.42825 |
| D35 | 1.364333 | 1.346833 | 1.414583 | 1.407625 |
| D56 | 1.258917 | 1.197417 | 1.28125 | 1.27575 |

### Discussion

For both Clostridial antigens the responses in cattle mirrored those obtained previously with sheep and a recombinant parasite antigen. Both the daily and the weekly progressively-increasing injections stimulated an immunity that was very similar that obtained by two antigenic exposures given 28 days apart.

Although there are at least three products available for simulating in one injection the two antigenic exposures (polylactide/coglycolide microbeads, latex-coated explo-tab, osmotic pump-driven intermittent exposure to antigen), none appear to have reached commercial success except for perhaps microbead presentation of Tetanus toxoid for single-shot human immunisation.

The experience of the inventors has shown that continuous first-order antigen delivery from implants is effective in stimulating some degree of immunity, and promotes memory for an anamnestic response to a further antigen exposure. However, the degree of immunity is matched to the release of antigen, and excessive amounts of antigen are required to maintain a protective immunity. When the antigen is used up the antibody levels revert to zero, although memory is still present.

Aspects of the present invention have been described by way of example only and it should be appreciated that modifications and additions may be made thereto without departing from the scope thereof as defined in the appended claims.

1. A process for administering one or more biologically active agents to an animal, comprising administering to said animal on a single occasion said one or more biologically active agents which in a form that releases progressively increasing doses of said one or more biologically active agents over a predetermined period of time.

2. A process as claimed in claim 1 wherein said one or more biologically active agents are selected from the group consisting of: antibiotics, antihelminthics, peptides, proteins, carbohydrates, DNA, RNA, hormones, neutraceuticals, vitamins, trace-elements, immunizing agents and any combination thereof.

3. A process as claimed in claim 1 wherein said one or more biologically active agents comprise an adjuvant and/or a pharmaceutically acceptable carrier.

4. A process as claimed in claim 1 wherein the progressively increasing doses comprise sequentially doubling doses of said biologically active agents.

5. A process as claimed in claim 1 wherein the predetermined period of time at which said one or more of biologically active agents are administered is selected from the group consisting of: hours, days, weeks, months and any combination thereof.

6. A process as claimed in claim 1 wherein said one or more biologically active agents is an immunizing agent and wherein said process comprises the administration to said animal of progressively increasing doses of one or more immunizing agents.

7. A process as claimed in claim 6 wherein said one or more immunizing agents comprise an antigen or combination of antigens.

8. A process as claimed in claim 6 wherein said one or more immunizing agents comprise a vaccine or combination of vaccines.

9. A process as claimed in claim 6 wherein said one or more immunizing agents are selected from the group consisting of: molecules that will induce protective immunity against a disease causing organism, molecules that will induce functional immunity, and any combination thereof.

10. A process as claimed in claim 6 wherein the progressively increasing doses of said one or more immunizing agents are in the range of from about 0.1 μg to 1000 μg.

11. (canceled)

12. A biologically active agent delivery composition comprising one or more biologically active agents whereby said composition is configured to deliver progressively increasing doses of said one or more biologically active agents to be released over a predetermined period of time from a single administration.

13. A biologically active agent delivery composition as claimed in claim 12 wherein said one or more biologically active agents are selected from the group consisting of: antibiotics, antihelminthics, peptides, proteins, carbohydrates, DNA, RNA, hormones, neutraceuticals, vitamins, trace-elements, immunizing agents and any combination thereof.

14. A biologically active agent delivery composition as claimed in claim 12 wherein said one or more biologically active agents comprise an adjuvant and/or a pharmaceutically acceptable carrier.

15. A biologically active agent delivery composition as claimed in claim 12 wherein the progressively increasing doses comprise sequentially doubling doses of biologically active agents.

16. A biologically active agent delivery composition as claimed in claim 12 wherein the predetermined period of time at which said one or more of biologically active agents are administered is selected from the group consisting of: hours, days, weeks, months and any combination thereof.

17. A biologically active agent delivery composition as claimed in claim 12 wherein said one or more biologically active agents is an immunizing agent agents.

18. A biologically active agent delivery composition as claimed in claim 17 wherein said one or more immunizing agents comprise an antigen or combination of antigens.

19. A biologically active agent delivery composition as claimed in claim 17 wherein said one or more immunizing agents comprise a vaccine or combination of vaccines.

20. A biologically active agent delivery composition as claimed in claim 17 wherein_said one or more immunizing agents are selected from the group consisting of: molecules that will induce protective immunity against a disease causing organism, molecules that will induce functional immunity and any combination thereof.

21. A biologically active agent delivery composition as claimed in claim 17 wherein the progressively increasing doses of said one or more immunizing agents are in the range of from about 0.1 μg to 1000 μg.

22. A biologically active agent delivery composition as claimed in claim 12 wherein the delivery composition comprises two or more types of microspheres or microparticles, wherein each type of microsphere or microparticle contains a different dose of one or more biologically active agents and wherein said microspheres or microparticles comprise biodegradable material which will degrade over a known time period so that the lowest dose of biologically active agents is released from a first type of microsphere or microparticle at a set time after administration, followed by the next highest dose of biologically active agents at the next predetermined time.

23. A biologically active agent delivery composition as claimed in claim 12 wherein the delivery composition is located within a bioerodable device that releases progressively increasing amounts of the biologically active agents as it erodes.

24. A biologically active agent delivery composition as claimed in claim 12 wherein the delivery composition is administered to an animal by way of injection, ingestion or implantation.

25. A biologically active agent delivery composition as claimed in claim 24 wherein the administration of the delivery composition takes place shortly after birth of an animal, or when maternally-derived antibody has decreased sufficiently for the young animal to be able to respond to the vaccination, and provides immunity without the need for further booster administration.

26. A multicompartmental capsule comprising a plurality of compartments with progressively increasing doses of one or more biologically active agents within the compartments, said capsule comprising an outer wall made of a at least one biodegradable substance which degrades over a pre-set period of time to release the smallest dose of biologically active agents, and one or more inner compartmental walls made of the same or different material that degrade over a longer period of time to release progressively increasing pulses of the biologically active agents

27. A capsule as claimed in claim 26 wherein the biodegradable material of the outer wall is selected from the group consisting of: cholesterol/lecithin, polylactide, polyglycolide copolymers, cellulose polymers, polyacrylic acid, polymethylmethacrylate, cross-linked polyacrylic acid, polyeaprolactone, polyvinylpyrrolidine, polyvinylalcohol, polyethylene glycol, agarose, DEAE dextran microspheres, starch microspheres, albumin microspheres, gelatin microspheres, and any combination thereof.

28. A capsule as claimed in claim 26 wherein the pre-set period of time within which the outer wall and inner compartmental walls degrade may be selected from the group consisting of: hours, days, weeks, and months.

29-30. (canceled)

31. The process of claim 4 wherein said sequentially increasing doses are 25, 50, 75, 100 or 4, 8, 32, 150 active units.

32. The process of claim 15 wherein said sequentially increasing doses are 25, 50, 75, 100 or 4, 8, 32, 150 active units.
